(19) United States
(12) Patent Application Publication
(10) Pub. No.: US 2006/0275815 A1
(43) Pub. Date: Dec. 7, 2006

(54) Method of and apparatus for the purification of nucleic acids using immobilized metal-ligand complex

(76) Inventors: Chang-eun Yoo, Seoul (KR); Sang-hyun Peak, Seoul (KR); Sook-young Kim, Yongin-si (KR); Jong-myeon Park, Seoul (KR)

Correspondence Address: CANTOR COLBURN, LLP, 55 GRIFFIN ROAD SOUTH, BLOOMFIELD, CT, 06002, US

(21) Appl. No.: 11/446,795

(22) Filed: Jun. 5, 2006

(30) Foreign Application Priority Data
Jun. 4, 2005 (KR) 10-2005-0048105

(51) Int. Cl. C12Q 1/68; C07H 21/04

(52) U.S. Cl. 435/6; 536/25.4; 435/287.2; 210/658; 210/659

(57) ABSTRACT

Provided is a method of purifying nucleic acids using a metal-ligand complex, the method comprising: immobilizing the metal-ligand complex on a solid support; bringing a sample containing the nucleic acid into contact with the immobilized complex on the solid support to bind the nucleic acid to the complex; and adding a solution containing a chelate capable of removing the metal to elute the nucleic acid bound to the complex. The nucleic acids can be efficiently purified using a metal-ligand complex interacting with a base of the nucleic acid instead of interacting with a phosphoric acid of the nucleic acid to selectively bind the nucleic acid, and using a chelator capable of removing the metal to elute the nucleic acid.

### CROSS-REFERENCE TO RELATED PATENT APPLICATION

This application claims the benefit of Korean Patent Application No. 10-2005-0048105, filed on Jun. 4, 2005, in the Korean Intellectual Property Office, the disclosure of which is incorporated herein in its entirety by reference.

### BACKGROUND OF THE INVENTION

1. Field of the Invention

The present invention relates to a method of and apparatus for the purification of nucleic acids using an immobilized metal-ligand complex.

2. Description of the Related Art

The isolation of DNA from cells has been performed using materials that have a proclivity for binding to DNA. Materials used for the isolation of DNA may be silica, glass fiber, anion exchange resin or magnetic beads (Rudi, K. et al., Biotechniqures 22, 506-511 (1997); and Deggerdal, A. et al., Biotechniqures 22, 554-557 (1997)). Several automatic apparatuses for the extraction of large quantities of DNA have been developed to avoid manual steps and remove operator's errors.

The production of high purity double-strand plasmid DNAs, single-strand phage DNAs, chromosomal DNAs, and agarose gel-purified DNA fragments is very important in molecular biology. Ideal methods of purifying DNA should be simple, able to be performed rapidly, and require little additional manipulation of samples. The DNAs obtained using such methods are ready for direct transformation, restriction enzyme analysis, ligation, or sequencing. Such methods are very attractive for the automated production of DNA samples, which is favored in research and diagnosis labs.

Conventional methods of purifying nucleic acids using a solid phase material are known. For example, U.S. Pat. No. 5,234,809 discloses a method of purifying nucleic acids using a solid phase material to which nucleic acids are bound. Specifically, the method includes mixing a starting material, a chaotropic material and a nucleic acid binding solid phase material; separating the solid phase material to which nucleic acid is bound from the liquid; and washing the solid phase material-nucleic acid complexes. However, this method is time consuming and complicated, and thus is not suitable for a Lab-On-a-Chip (LOC).

U.S. Pat. No. 6,291,166 discloses a method of archiving nucleic acids using a solid phase matrix. This method is advantageous in that, since nucleic acids are irreversibly bound to the solid phase matrix, a delayed analysis or repeated analysis of the nucleic acid solid phase matrix complexes is possible. However, according to this method, aluminum (Al) which has a positively-charged surface should be rendered hydrophilic with basic materials, such as NaOH, and nucleic acids are irreversibly bound to the Al rendered hydrophilic, and thus cannot be separated from the Al.

U.S. Patent Publication No. 2004/0152076 discloses a method of extracting a target gene using metal affinity chromatography including immobilized metal ions binding to a base of the gene. Although the use of binding between a metal-ligand complex and a base of nucleic acids is described, there is no description of a method of eluting the nucleic acids using a chelator capable of removing the metal after forming a complex for purifying nucleic acids.

The inventors of the present invention discovered that the nucleic acid can be effectively purified by binding a metal-ligand complex having a specific interaction with the base of the nucleic acid to the nucleic acid and adding a chelator into a metal-ligand complex bound with the nucleic acids to elute the nucleic acid.

### SUMMARY OF THE INVENTION

The present invention provides a method of purifying nucleic acids using a metal-ligand complex. The method includes immobilizing a metal-ligand complex on a solid support, bring a sample containing nucleic acids into contact with the immobilized complex on the solid support to bind the nucleic acids with the complex, and adding a solution containing a chelate capable of removing the metal to elute the nucleic acids bound to the complex.

The present invention also provides an apparatus for purifying nucleic acids including a chelator solution capable of removing a metal and a solid support having an immobilized metal-ligand complex.

According to an aspect of the present invention, there is provided a method of purifying nucleic acids using a metal-ligand complex, the method including immobilizing a metal-ligand complex on a solid support, bringing a sample containing nucleic acids into contact with the immobilized complex on the solid support to bind the nucleic acids with the complex, and adding a solution containing a chelate capable of removing the metal to elute the nucleic acids bound to the complex.

The nucleic acid can be effectively purified by binding the metal-ligand complex specifically interacting with a base of a nucleic acid with the nucleic acid and adding a chelator to the metal-ligand complex bound with the nucleic acid to elute the nucleic acid. Thus, the nucleic acid can be effectively purified by immobilizing a high concentration of the metal-ligand complex on the chip. FIG. 1 is a schematic diagram illustrating an example of nucleic acid purification according to an embodiment of the present invention.

After the ligand is immobilized on the solid support, a metal ion is coordinated with the ligand. The immobilizing of the metal-ligand complex on the solid support can be performed using a known technique. For example, a silicon wafer may be used as the solid support. FIG. 2 is a schematic view illustrating the immobilizing of a Zn-cyclen complex on a silicon wafer. When the metal-ligand complex is immobilized on a flat substrate, the small surface area of the substrate allows only a small amount of the metal-ligand complex to bind to the solid support, thus limiting the contact with the nucleic acids. Therefore, the amount of the immobilized metal-ligand complex and the contact area between the solid support and the nucleic acids may be increased by immobilizing the metal-ligand on beads instead of the substrate.

In the method, a sample containing the nucleic acid is brought into contact with an immobilized complex on the solid support to bind the nucleic acid with the complex. Since the conventional methods use negative charges of the phosphoric acid in the nucleic acids, it is disadvantage in that all moieties with negative charges within the nucleic acid are bound to the complex, and thus the selectivity of the method is deteriorated. However, since the metal-ligand complex in an embodiment of the present invention interacts with the base of the nucleic acid instead of the phosphoric acid, the selectivity of the nucleic acid purification can be improved. Cu-iminodiacetic acid (Cu-IDA) is bound to an aromatic nitrogen of a base in single strand RNA and DNA (Biotechnol. Prog. 2003, 19: 982). Zn-cyclen is selectively bound to thymine in double strand DNA (Pure & Appl. Chem. 1997, 69: 2187). FIG. 3 is a schematic diagram illustrating that Zn-cyclen is bound to double-strand DNA. Therefore, in an embodiment of the present invention, the metal-ligand complex selectively bound to the double strand DNA is used.

The method further includes adding a solution containing a chelator capable of removing the metal to elute the nucleic acid bound to the complex. The nucleic acids are bound to the complex by the metal ion, and the chelator capable of removing the metal is added to the complex to elute the nucleic acid. In the elution of the nucleic acid, exchanging ligands can be considered in addition to using the chelator.

The method may further include washing the sample after binding the nucleic acid with the complex to remove uncombined portions of the sample. The nucleic acid can be purified more effectively by washing the sample and removing the uncombined portions of the sample after binding the nucleic acid with the metal-ligand complex. A solution used for binding the nucleic acid with the metal-ligand complex may be used for the washing.

The solid support may be a glass slide, a silicon wafer, magnetic beads, polystyrene, a membrane, or a metal plate. The solid support can be anything on which the metal-ligand complex can be immobilized. The solid support may be insoluble in water. When the solid support is soluble in water, it is difficult to separate the solution containing the nucleic acid from the solid support after the nucleic acid purification. The solid support may have a large surface area because more metal-ligand complexes can be bound thereto. To enlarge the surface area, a flat substrate, such as glass or a wafer, can be surface-processed in a pillar form.

The metal of the metal-ligand complex may be a transition metal ion, such as Cu (II), Co (III), Ni (II), Zn (II), or Fe (III). However, the present invention is not limited thereto, and any metal capable of forming a complex with a ligand can be used.

The ligand of the metal-ligand complex may be a cyclic polyamine compound such as cyclen, an aliphatic polyamine compound such as tris-(2-aminoethylamine), or a polycarboxy compound such as EDTA. A ligand generally refers to an ion or molecule bound to a central atom in a complex compound, which is a compound having a lone pair. For example, in [Co(NH₃)₆]Cl₃, K₃[FeCl₆], [Cu(NH₂CH₂COO)₂] etc., NH₃, Cl⁻, and NH₂CH₂COO⁻ etc. are ligands, being ions or molecules bound to central atoms Co³⁺, Fe³⁺, Cu²⁺ etc., respectively. The ligand may be a single atomic ion or a polyatomic group.

The chelator may be EDTA or HEDTA. The chelator can be anything which has a higher coupling constant for the metal ion than the ligand of the metal-ligand complex to elute the nucleic acid by separating the metal used for the metal-ligand complex from the ligand.

The contact between the sample and the complex is performed under static or fluidic conditions. The nucleic acid can be brought into contact with the solid substrate both under static and fluidic conditions. The floating solution including the nucleic acid is brought into contact with the solid substrate in a fluidic control system. In the fluidic control system, while the solid substrate may be flat, the substrate may have a plurality of pillars to increase the opportunity of contact between the nucleic acids and the solid substrate for more binding.

According to another aspect of the present invention, there is provided an apparatus for purifying nucleic acids that includes a chelator solution capable of removing the metal; and a solid support having the immobilized metal-ligand complex.

The chelator solution is capable of eluting the nucleic acid by removing the metal from the nucleic acid bound to the metal-ligand immobilized on the solid substrate. The nucleic acid can be effectively purified using the apparatus by supplying the sample containing the nucleic acid to the apparatus, binding the nucleic acid to the solid support immobilizing the metal-ligand complex and eluting the nucleic acid with the chelator solution.

In an embodiment of the present invention, the solid support may have the structure of a plate or a pillar. A solid support having a large surface area is advantageous in that more metal-ligand complexes can be bound therewith. To enlarge the surface area of the solid support, the surface of a flat solid support such as a glass or wafer support may be processed into pillars.

The solid support may be a glass slide, a silicon wafer, magnetic beads, polystyrene, a membrane, or a metal plate. The solid support can be anything on which the metal-ligand complex can be immobilized. The solid support may be insoluble in water. When the solid support is soluble in water, it is difficult to separate the solution containing the nucleic acid from the solid support after the nucleic acid purification.

The metal of the metal-ligand complex may be a transition metal ion such as Cu (II), Co (III), Ni (II), Zn (II), or Fe (III). However, the present invention is not limited thereto, and any metal capable of forming a complex with a ligand can be used.

The ligand of the metal-ligand complex may be a cyclic polyamine compound such as cyclen, an aliphatic polyamine compound such as tris-(2-aminoethylamine), or a polycarboxy compound such as EDTA.

The chelator may be EDTA or HEDTA. The chelator can be anything which has a higher coupling constant for the metal ion than the ligand of the metal-ligand complex to elute the nucleic acid by separating the metal used for the metal-ligand complex from the ligand.

### BRIEF DESCRIPTION OF THE DRAWINGS

The above and other features and advantages of the present invention will become more apparent by describing in detail exemplary embodiments thereof with reference to the attached drawings in which:

FIG. 1 is a schematic diagram illustrating nucleic acid purification according to an embodiment of the present invention;

FIG. 2 is a schematic diagram illustrating the immobilization of a Zn-cyclen complex on a silicon wafer; and

FIG. 3 is a schematic diagram illustrating Zn-cyclen bound to double strand DNA.

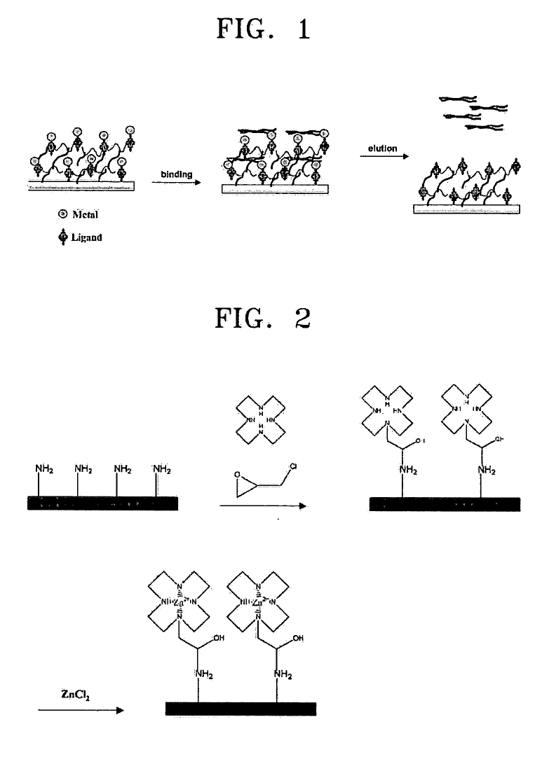

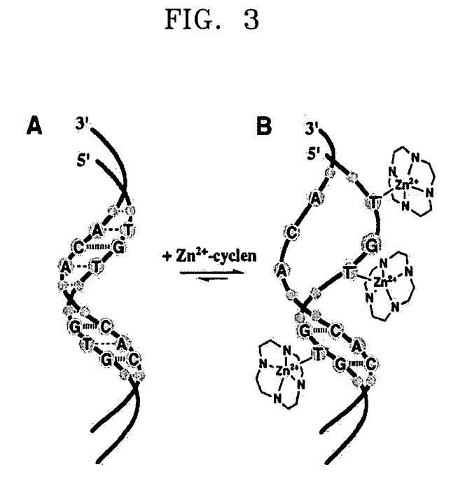

### DETAILED DESCRIPTION OF THE INVENTION

The present invention will now be described in greater detail with reference to the following examples. The following examples are for illustrative purposes only and are not intended to limit the scope of the invention.

### EXAMPLES

### Example 1

### Immobilization of a Metal-Ligand Complex on a Substrate

A metal-ligand complex was immobilized on a silicon wafer coated with γ-aminopropylsilane used as a substrate activated with an amino group. 0.5 g of cyclen as a ligand and 100 μl epichlorohydrin were dissolved in 5 ml of DMF, and then the resulting solution was applied to the silicon wafer to immobilize the ligand on the substrate. After 0.1 g of ZnCl₂ was dissolved in DMF, the solution was applied to the substrate to prepare a Zn-cyclen complex.

### Example 2

### Binding and Eluting Effects of a Zn-cyclen Complex Immobilized to the Substrate

Genome DNA of E. coli HB101 was used for the Zn-cyclen complex to investigate binding and eluting effects of the Zn-cyclen complex immobilized to the substrate. First, genome DNA of E. coli HB101 was dissolved in a binding solution (Tris 5 mM, NaCl 10 mM, pH 7.6). 15 μl of the genome DNA solution was injected into a Zn-cyclen complex immobilized on a substrate. The binding between the genome DNA and the Zn-cyclen complex was performed for 3 minutes. The solution was removed and the substrate was washed three times with 15 μl of the binding solution. Then, the elution was performed using nucleic acid elution buffers. 15 μl of a buffer (Tris (10 mM), EDTA (10 mM), pH8.4) was used to remove the metals by EDTA, and 15 μl of a buffer (histidine (50 mM), pH 7.4) was used to exchange ligands with histidine.

The eluting effects according to the two buffers are shown in Table 1.

TABLE 1

| | Zn-cyclen |
|---|---|
| Binding efficiency | 2.78% |
| Elution efficiency 1 (removing metals) | 54.12% |
| Elution efficiency 2 (exchanging ligands) | 0% |

As shown in Table 1, while the genome DNA was not eluted by exchanging ligands with hidtidine, the genome DNA was eluted by removing metal ions with EDTA. The binding efficiency of the nucleic acids is believed to be low since the surface of the solid substrate was flat. Thus, the binding efficiency is expected to be greater when a pillar structure is used.

Therefore, the nucleic acid can be eluted effectively using the chelator such as EDTA.

### Example 3

### Binding and Eluting Effect of the Nucleic Acid in a Fluidic Control Condition

In a fluidic control system, the binding and elution of nucleic acid were investigated. First, genome DNA of E. coli HB101 was dissolved in a binding solution (Tris 5 mM, NaCl 10 mM, pH 7.6) at a concentration of 11 ng/μl. The genome DNA solution was injected into the Zn-cyclen complex immobilized substrate at a flow rate of 40 μl/min for four minutes using an injection pump (HARVARD, PHD2000). Then, the substrate was washed with the binding solution at a flow rate of 40 μl/min for two minutes. The elution was performed using nucleic acid elution buffers (Tris (10 mM), EDTA (10 mM), pH 8.4 and Tris (10 mM), EDTA (10 mM), pH 9.3) at a flow rate of 40 μl/min for 8 minutes. As a comparative example, a substrate immobilizing cyclen not coordinated with Zn and a solid substrate having a charge reversible surface (CRS) (disclosed in Korean Patent Application No. 2004-0111165, herein incorporated by reference in its entirety) were used. In the elution of the CRS substrate, Tris (10 mM), pH 9.0 was used.

The elution effects according to the two buffers are shown in Table 2.

TABLE 2

| | Zn-cyclen(%) | cyclen | CRS |
|---|---|---|---|
| Biding efficiency | 13.07 | 19.28 | 21.40 |
| Elution efficiency 1 | 38.00 | 20.91 | 57.32 |
| (TE 10 mM, pH8.4) | | | (Tris 10 mM, pH9.0) |
| Elution efficiency 2 | 54.56 | 24.99 | |
| (TE 10 mM, pH9.3) | | | |

As shown in table 2, the cyclen not having Zn had a higher binding efficiency than the Zn-cyclen. However, the Zn-cyclen had a higher elution efficiency than the cyclen. The elution of the Zn-cyclen remarkably increased as the pH increased, but the elution of the cyclen did not significantly increase. The substrate using CRS had a higher binding efficiency than the Example of the present invention. The substrate using CRS and the Example of the present invention had similar elution efficiency. Thus, EDTA in the elution buffer effectively elutes the nucleic acid by removing the metals.

According to the present invention, nucleic acids can be efficiently purified using a metal-ligand complex interacting with a base of the nucleic acid instead of interacting with a phosphoric acid of the nucleic acid to selectively bind the nucleic acid, and using a chelator capable of removing the metal to elute the nucleic acid.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A method of purifying nucleic acids using a metal-ligand complex, the method comprising:
immobilizing the metal-ligand complex on a solid support;
bringing a sample containing the nucleic acid into contact with the immobilized complex on the solid support to bind the nucleic acid to the complex; and
adding a solution containing a chelate capable of removing the metal to elute the nucleic acid bound to the complex.

2. The method of claim 1, further comprising washing the sample after binding the nucleic acid to the complex to remove uncombined portions of the sample.

3. The method of claim 1, wherein the solid support is selected from the group consisting of slide glass, silicon wafer, magnetic bead, polystyrene, membrane and metal plate.

4. The method of claim 1, wherein the metal of the metal-ligand is a transition metal ion selected from the group consisting of Cu (II), Co (III), Ni (II), Zn (II), and Fe (III).

5. The method of claim 1, wherein the ligand of the metal-ligand complex is selected from the group consisting of a cyclic polyamine compound including cyclen, an aliphatic polyamine compound including tris-(2-aminoethylamine), and a polycarboxy compound including EDTA.

6. The method of claim 1, wherein the chelator has a higher coupling constant for the metal ion than the ligand of the metal-ligand complex, and is one selected from the group consisting of EDTA and HEDTA.

7. The method of claim 1, wherein the contact between the sample and the complex is performed under static or fluidic conditions.

8. An apparatus for purifying nucleic acids, the apparatus comprising:
a chelator solution capable of removing a metal; and
a solid support having an immobilized metal-ligand complex.

9. The apparatus of claim 8, wherein the solid support has a pillar structure.

10. The apparatus of claim 8, wherein the solid support is selected from the group consisting of a glass slide, a silicon wafer, magnetic beads, polystyrene, a membrane and a metal plate.

11. The apparatus of claim 8, wherein the metal of the metal-ligand is a transition metal ion selected from the group consisting of Cu (II), Co (III), Ni (II), Zn (II), and Fe (III).

12. The apparatus of claim 8, wherein the ligand of the metal-ligand complex is selected from the group consisting of a cyclic polyamine compound including cyclen, an aliphatic polyamine compound including tris-(2-aminoethylamine), and a polycarboxy compound including EDTA.

13. The apparatus of claim 8, wherein the chelator has a higher coupling constant for the metal ion than the ligand of the metal-ligand complex and is one selected from the group consisting of EDTA and HEDTA.
